(19) United States
(12) Patent Application Publication
(10) Pub. No.: US 2025/0316369 A1
(43) Pub. Date: Oct. 9, 2025

(54) DIGITAL TRIAGE AND EMERGENCY RESPONSE, RESPONDER, AND PATIENT BIOMONITORING AND TRACKING METHOD AND SYSTEM

(71) Applicant: Inhance Digital Corporation, Los Angeles, CA (US)

(72) Inventors: Maziar Farzam, Los Angeles, CA (US); Brian Moore, Los Angeles, CA (US)

(73) Assignee: Inhance Digital Corporation, Los Angeles, CA (US)

(21) Appl. No.: 19/174,424

(22) Filed: Apr. 9, 2025

Related U.S. Application Data
(60) Provisional application No. 63/631,865, filed on Apr. 9, 2024.

(51) Int. Cl. G16H 40/20; G16H 10/60; G16H 15/00; G16H 40/67; G16H 50/20

(52) U.S. Cl. CPC G16H 40/20; G16H 10/60; G16H 15/00; G16H 40/67; G16H 50/20

(57) ABSTRACT

The present invention provides a method and system for Digital Patient Tracking and Triage System (DPTTS) that enables next-generation digital medical triage, specifically allowing first responders to transmit patients' biomedical data, utilizing wearable biosensors, to respective medical/emergency care facilities. The present invention also provides a novel method of medical triage and patient tracking that bridges the gap from incident/emergency to the medical/emergency care facility while integrating wearable biosensors and IoT devices to measure and transmit the patient's biomedical data. It visually integrates this data into a common operating picture to provide situational awareness in medical emergencies to empower EMTs and other emergency responders, particularly in mass casualty and disaster events.

### CROSS-REFERENCE TO RELATED APPLICATIONS

This application claims priority to and the benefit of U.S. Provisional Patent Application No. 63/631,865, filed Apr. 9, 2024, entitled “METHOD AND SYSTEM FOR DIGITAL TRIAGE AND EMERGENCY RESPONSE, FIRST RESPONDER AND PATIENT BIOMONITORING AND TRACKING,” the entire contents of which are incorporated herein by reference.

### BACKGROUND OF THE INVENTION

### 1. Field of Invention

The invention relates to a computer information management, processing, and display system to overcome the technological problem of tracking, prioritizing, and monitoring persons, some of whom may be in dire need of immediate attention, over a network in large scale emergency medical situations. The system is a real-world tool that is particularly useful for both emergency first responders and patients and bridges a communications gap between an incident or emergency location and a medical/emergency care facility.

### 2. Description of Related Art

Current computer systems for emergency medical response face significant technological limitations in their ability to process, track, and transmit critical patient data during mass casualty events. The fundamental technical problem lies in the lack of robust digital infrastructure capable of capturing, processing, and securely transmitting patient vital signs from emergency scenes to healthcare facilities in real-time.

Existing computer implementations rely on disconnected local computing devices that store patient data in isolation, without the capability to integrate or share this information across a unified digital platform. This creates a critical technical gap where patient data such as temperature, pulse rate, respiratory rate, blood pressure, oxygen saturation, and electrocardiogram, among other vitals, even when captured electronically at an incident scene by physiological monitoring devices remains siloed on local devices and cannot be efficiently transmitted to emergency care facility computer systems for advance medical planning. The technical challenge is further complicated by the need to implement secure, HIPAA-compliant data transmission protocols while maintaining real-time communication capabilities in emergency environments.

Current computer systems lack the technical architecture necessary to enable automated digital hand-off of patient data from point-of-injury through the entire care continuum. The absence of an integrated digital platform that can combine vital signs monitoring, geolocation tracking, and secure data transmission creates significant technical barriers to effective patient care. In battlefield and combat scenarios, the technical limitations of existing computer systems are even more pronounced. Current implementations are incapable of automated vital signs tracking during battlefield evacuation and transport from point of injury to combat support hospitals (CSH) or field hospitals (FH). Combat medics rely on paper-based DD Form 1380 triage tags with no ability to digitally process and transmit this critical data. Current computer implementations cannot provide the real-time situational awareness and data analytics capabilities needed for modern emergency response.

The present invention addresses these specific technical problems through an innovative computer architecture that enables seamless digital capture, processing, analysis, and secure transmission of critical patient data across the emergency response ecosystem.

### SUMMARY OF THE INVENTION

The present invention overcomes these challenges and other deficiencies of the prior art by providing a computer network method and system for digital emergency triage and patient tracking that overcome the limitations of conventional approaches. The present invention is a Digital Patient Tracking and Triage System (DPTTS) implementing comprehensive emergency response capabilities through multiple integrated subsystems. The system enables patient monitoring and triage classification using the DIME framework (Delayed, Immediate, Minor, Expectant) through a centralized software platform accessible to firefighters, EMTs, police, and federal/state emergency operators.

The implementation tracks patient and emergency responder locations through mobile device geolocation services, transmitting position data to cloud infrastructure via a DPTTS mobile application. The system simultaneously monitors patient and responder biometric data including heart rate, blood pressure, and oxygen saturation through integrated wearable sensors, providing real-time health status updates to both field personnel and incident commanders.

The system processes multimedia data streams, including digital images and live video from mobile devices, implementing secure encryption protocols for transmission between emergency responders and command dashboards. Real-time incident visualization occurs through three-dimensional mapping interfaces, supporting multiple extended reality platforms (AR, VR, MR, XR) to enhance situational awareness for command personnel.

The implementation incorporates multiple video capture sources, including unmanned aerial vehicles, helmet-mounted cameras, and body-worn devices. This multi-source video architecture enables comprehensive incident documentation while maintaining secure transmission pathways between field personnel and command infrastructure through the DPTTS dashboard interface.

The implementation captures and processes critical patient data through multiple channels. When a patient enters the system, their vital information is digitally recorded and tracked, including heart rate, blood pressure, oxygen saturation levels, and other key biometric indicators through wearable biosensors. This data streams continuously to both emergency responders and medical facilities, enabling proactive patient care management.

The system implements automated patient identification and tracking capabilities through various technologies including barcode scanning, NFC, and RFID tags. This ensures accurate patient tracking throughout the emergency response process while maintaining secure storage of patient information. The implementation includes computer vision algorithms for processing patient identification documents and maintaining continuity of care even for unidentified patients.

For enhanced patient monitoring, the system generates real-time visualizations of patient health trends through gradient mapping and predictive analytics. The implementation utilizes machine learning algorithms to analyze patient biometric data patterns and predict potential health deterioration, enabling faster medical intervention. This capability allows emergency responders and medical staff to identify patients requiring immediate attention based on declining vital signs or concerning health trends.

The system maintains HIPAA compliance through specialized data de-identification protocols during transmission, ensuring patient privacy while enabling critical information sharing between emergency responders and healthcare facilities. This secure data handling enables seamless integration with electronic health records (EHR) systems while protecting sensitive patient information.

In an embodiment of the invention, a system for digital emergency triage and tracking, comprises: an incident map generator configured to generate a real-time digital map of an emergency incident; a biometrics generator configured to collect and transmit one or more vital signs of one or more patients; an incident navigator configured to provide a points of interest (POI) interface; a tagging manager configured to associate digital identifiers with the one or more patients; and a data collector and analyzer configured to process and analyze the vital signs of the one or more patients, wherein the system is configured to digitally track and monitor triage states and locations of the one or more patients. The incident map generator is configured to generate a model of an emergency environment using geographical information system (GIS) data. The one or more vital signs are selected from the group consisting of: heart rate, blood pressure, oxygen saturation, respiration rate, electrocardiogram, electroencephalogram, body temperature, and a combination thereof. The POI interface is configured to display a virtual representation of an emergency location, the one or more patients, and one or more first responders. The digital identifiers are associated with physical triage tags. The data collector and analyzer is configured to: determine the triage states of the one or more patients. The system may further comprise an incident commander dashboard configured to display aggregated data associated with the one or more patients and the real-time digital map of the emergency incident data using at least one of augmented reality, virtual reality, or mixed reality technologies.

In another embodiment of the invention, a method for digital emergency triage and tracking, comprises: generating a virtual incident environment; registering and tagging a plurality of patients with digital identifiers; collecting vital signs using wearable sensors of the plurality of patients; analyzing the collected vital signs to determine triage states of the plurality of patients; tracking locations of the plurality of patients; and outputting the collected vital signs and the determined triage states to an emergency care facility. The virtual incident environment comprises accessing geographical information system data; generating a two-dimensional or three-dimensional model; adding one or more virtual points of interest; and providing a navigation interface. Collecting vital signs using wearable sensors of the plurality of patients comprises measuring at least one of: heart rate, blood pressure, oxygen saturation, respiration rate, electrocardiogram, electroencephalogram, body temperature. The registering and tagging the plurality of patients comprises scanning physical triage tags; capturing patient identification information; and storing patient data in a database. The analyzing the collected vital signs to determine triage states comprises processing the collected vital signs; determining triage states using predefined algorithms; generating data visualizations; and updating patient status in real-time. The method may further comprise displaying aggregated incident data through an incident commander dashboard using extended reality technologies.

In yet another embodiment of the invention, a non-transitory computer-readable medium storing instructions that, when executed by a processor, cause the processor to perform operations comprising generating a virtual incident environment; managing digital patient triage tags; collecting and analyzing patient vital signs; tracking patient locations; and providing real-time incident awareness. The operations may further comprise displaying patient vital signs on virtual avatars; updating triage states in real-time; generating heat maps of patient data; and providing predictive analytics using machine learning. The managing digital patient triage tags comprises scanning physical triage tags; associating digital identifiers with patients; storing triage states; and updating patient status.

In another embodiment of the invention, a wearable device for patient monitoring during emergency response, comprises: one or more biometric sensors configured to measure one or more patient vital signs; a location tracking module; a wireless communication interface; and a processor configured to transmit patient data to a digital triage system. The one or more biometric sensors comprise at least one of: a heart rate sensor; a blood pressure sensor; an oxygen saturation sensor; a respiration rate sensor; and a temperature sensor.

In yet another embodiment of the invention, a system for emergency incident command, comprises: a holographic display interface; a virtual incident map; real-time patient tracking; biometric data visualization; and predictive analytics capabilities. The system may further comprise eye-tracking hardware configured to monitor commander interactions; heat mapping functionality for patient data analysis; and machine learning algorithms for health event prediction.

Key advantages of the present invention include seamless digital tracking of patient triage states and locations, automated collection and analysis of vital signs data, real-time transmission of patient information to care facilities, enhanced situational awareness for incident commanders, secure, HIPAA-compliant data handling, and integration with existing electronic health record systems.

The invention represents a significant advance in emergency medical response capabilities by providing a comprehensive digital solution that bridges the critical information gap between incident locations and emergency care facilities.

The foregoing and other features and advantages of the invention will be apparent from the following more detailed description of the invention's preferred embodiments and the accompanying drawings.

### BRIEF DESCRIPTION OF THE DRAWINGS

For a complete understanding of the present invention and advantages thereof, reference is now made to the ensuing descriptions taken in connection with the accompanying drawings briefly described as follows:

FIG. 1 illustrates a Digital Patient Triage and Tracking System according to an embodiment of the invention;

FIG. 2 illustrates a process for digital patient triage and tracking according to an embodiment of the invention;

FIG. 3 illustrates a computer system implementing specialized hardware and software components to enable digital triage and emergency response capabilities according to an embodiment of the invention;

FIG. 4 illustrates a chip set that can be used to implement an embodiment of the invention;

FIG. 5 is an exemplary diagram of a mobile station (e.g., handset) that can be used to implement an embodiment of the invention;

FIG. 6 is an exemplary diagram of a system architecture that can be used to implement an embodiment of the invention; and

FIG. 7 is an exemplary data flow diagram that can be used to implement an embodiment of the invention.

### DETAILED DESCRIPTION OF PREFERRED EMBODIMENTS

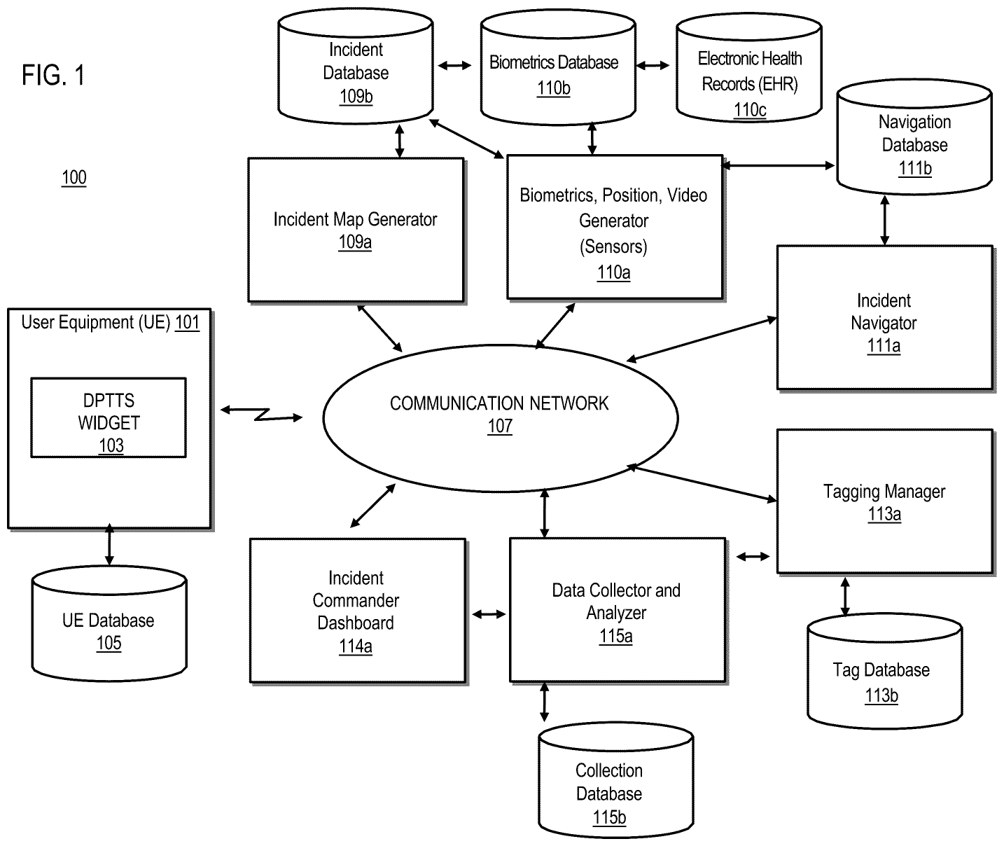

Preferred embodiments of the present invention and their advantages may be understood by referring to FIGS. 1-7. The described features, structures, or characteristics of the invention may be combined in any suitable manner in one or more embodiments. It will be apparent to those skilled in the art that modifications and variations can be made to the present invention without departing from the invention's spirit and scope. Thus, it is intended that the current invention cover all modifications and variations consistent with the scope of the appended claims and their equivalents.

The invention addresses critical technological needs in emergency medical response while providing innovative solutions through its Digital Patient Triage and Tracking System (DPTTS). The DPTTS is capable of storing, tracking, and updating patient “DIME” status while providing accurate positional tracking from incident scenes to hospitals. DIME is the triage classification system used in conventional emergency medical response that stands for: Delayed (yellow): Patients requiring observation and possible later re-triage. Their medical condition is stable and not in immediate danger. Immediate (red): Patients who cannot survive without immediate treatment but have a chance of survival. Minor (green): “Walking wounded” patients who will need medical care later after more critical injuries are treated. Expectant (black): Deceased patients and those with extensive injuries who will not survive given the available care. This system is traditionally implemented through color-coded paper tags that emergency medical technicians (EMTs) attach to patients during mass casualty events to visually indicate their injury level and treatment priority.

The DPTTS system implements advanced sensing, tracking, and data science capabilities to enhance emergency response operations. The system architecture combines specialized hardware and software components including biosensors, motion tracking, and location monitoring to provide comprehensive situational awareness. The implementation enables real-time patient tracking, biometric monitoring, and virtual environment generation while supporting artificial intelligence and machine learning-enabled predictive analytics for identifying critical health trends.

FIG. 1 illustrates a DPTTS system 100 according to an embodiment of the invention. The system 100 comprises one or more user equipment (UE) 101 having connectivity to an incident map generator 109a; a biometrics, position, and video generator 110a; an incident navigator 111a; a tagging manager 113a; and a data collector and analyzer 115a via a communication network 107. In various embodiments, these hardware components can be implemented through specific technical configurations to enable the digital triage and tracking functionality. The system utilizes specialized computing equipment including mobile terminals, fixed terminals, and portable terminals configured with specific processing capabilities for emergency response applications. These terminals can be augmented with dedicated wearable biosensors and monitoring devices that collect and transmit vital patient data through secure network protocols. The display systems incorporate both traditional direct-view technologies and advanced holographic interfaces, enabling real-time visualization of incident data through various extended reality platforms. The network infrastructure implements multiple wireless and fixed communication protocols to ensure reliable data transmission across emergency environments. Scanning hardware integrates with the system through standardized interfaces, while dedicated command center equipment provides comprehensive monitoring and control capabilities. The following detailed description provides specific technical implementations of these components and their integration within the overall system architecture.

The communication network 107 comprises one or more network types implemented through various hardware and software configurations. The network infrastructure includes data networks that implement packet-switched protocols through local area networks for facility-level connectivity, metropolitan area networks for city-wide coverage, wide area networks for regional connectivity, and Internet connectivity for global reach. The system further utilizes proprietary cable and fiber-optic networks to enable secure data transmission. In wireless embodiments, the system may be implemented on cellular networks supporting Enhanced Data rates for Global Evolution, General Packet Radio Service, Global System for Mobile Communications, Internet Protocol Multimedia Subsystem, and Universal Mobile Telecommunications System protocols. Additional wireless capabilities are enabled through Worldwide Interoperability for Microwave Access, Long Term Evolution, and Code Division Multiple Access technologies. The system further implements supplementary wireless capabilities through Wi-Fi networks for local wireless connectivity, satellite and satellite internet systems for remote coverage, and mobile ad-hoc networks for dynamic battlefield environments. This comprehensive network architecture enables seamless integration between these various communication technologies to provide robust coverage and redundancy for emergency response operations. The network implementation utilizes standardized protocols across the Open Systems Interconnection model layers, from physical signal generation through application-level data processing. This enables efficient packet-based data exchange between network nodes while maintaining secure and reliable communication channels for emergency response coordination.

The user equipment (UE) 101 implements multiple computing platforms including but not limited to: mobile terminals, fixed terminals, portable terminals, extended reality devices, smart wearables, neural interfaces, quantum computing interfaces, and future computing paradigms. The system architecture supports seamless integration of emerging technologies while maintaining backward compatibility with existing emergency response infrastructure.

The incident map generator 109a comprises specialized hardware and software components for creating dynamic emergency response visualizations. The generator 109a implements a map generation engine that connects to incident database 109b through communication network 107. This engine generates both two-dimensional and three-dimensional models specifically configured for emergency and disaster environment representation.

The map generation process utilizes comprehensive geospatial data sources including but not limited to: GIS platforms, satellite imagery, LIDAR scanning, photogrammetry, real-time sensor networks, crowd-sourced mapping data, and autonomous mapping systems. The implementation supports dynamic integration of multiple data streams while enabling real-time environmental updates. The generator 109a integrates these data sources with specialized 2D/3D map datasets and wireframe models stored in incident database 109b. Generator 109a implements advanced rendering capabilities to modify these base models through the addition of textures, materials, virtual cameras, and lighting effects for structures and terrain features. This processed virtual environment data is then transmitted to UE 101 and DPTTS widget 103 through secure network protocols.

The virtual incident environment implementation creates precise digital representations of actual emergency scenes. The system generates detailed models of infrastructure including virtual buildings, roads, and natural land features such as mountains, hills, rivers, and vegetation. The incident environment generator further implements virtual points of interest (POIs) as interactive elements within the rendered space. A key technical feature includes a dynamic virtual camera system that automatically tracks first responders' virtual avatars as they navigate through the actual environment. This allows the virtual environment to precisely mirror real-world emergency locations-for example, creating a virtual model of a city's physical downtown environment with specific geolocation, buildings, and landmarks specific to that location.

Building on the virtual environment capabilities, the system 100 implements advanced rendering and interaction mechanisms through multiple technical approaches. UE 101 utilizes a comprehensive suite of application programming interfaces (APIs) for generating and displaying virtual incident environments. The rendering engine specifically implements Web3D technologies including WebGL, OpenGL, OpenGL ES, OpenXR, and WebXR APIs. The system architecture supports additional rendering frameworks including Direct3D, Vulkan, Metal, and numerous specialized 3D graphics engines to ensure compatibility across different computing platforms.

The system enables streamlined incident response initiation through web-based access protocols. First responders can activate the system by accessing a designated URL through DPTTS widget 103. Upon system initialization, first responders receive incident assignments through the Incident Commander interface, enabling access to relevant points of interest through the virtual navigation system. The implementation supports hybrid operation modes that combine virtual and physical environment interactions.

In physical deployment scenarios, the system utilizes machine-readable indicators including QR codes, barcodes, RFID, and NFC tags to associate digital information with physical objects and patients. When a first responder scans these indicators using UE 101 or DPTTS widget 103, the system automatically renders corresponding virtual environment elements. This creates an augmented operational environment where first responders can simultaneously interact with both physical elements and their virtual representations, enhancing situational awareness and response capabilities.

The system further implements avatar-based representation of first responders within the virtual environment, displaying personnel as customizable 2D or 3D models. These avatar configurations can be managed through incident commander 114a or individually modified via DPTTS widget 103, enabling clear visual identification, and tracking of response personnel throughout the incident space.

The DPTTS widget 103 comprises a sophisticated user interface component that implements multiple critical functionalities for emergency response operations. The widget enables modification and interaction with virtual environments through an advanced interface that allows customization of textures, materials, appearance, layout, and other visualization attributes. The system implements secure access controls through an authentication framework that requires user credential validation. This authentication system interfaces directly with tagging manager 113a to ensure proper security protocols are maintained during emergency operations.

The widget 103 includes comprehensive scanning functionality that processes Near-Field Communication tags, Radio Frequency Identification tags, and various optical codes including barcodes and QR codes. The implementation includes a sophisticated graphical targeting system with real-time video feed integration to ensure precise scanning alignment during emergency operations. The widget's environment interaction capabilities enable first responders to navigate seamlessly through both virtual and physical incident spaces while accessing critical incident data through web-based interfaces. The system supports avatar customization features that integrate with incident commander 114a for personnel tracking and identification.

Through this integrated architecture, the widget 103 enables enhanced situational awareness by supporting both virtual and augmented reality implementations, allowing first responders to maintain comprehensive awareness of both physical and virtual elements within the emergency response environment.

Building on the DPTTS widget capabilities, the system implements comprehensive biometric monitoring and data collection functionality through specialized hardware and software components. The biometrics/position/video generator 110a connects to biometrics database 110b through communication network 107 to enable real-time health monitoring during emergency operations.

The generator 110a implements advanced biosensor integration to collect critical patient vital signs including heart rate, blood pressure, oxygen saturation, respiration rate, electrocardiogram data, and core body temperature. This biometric data collection occurs through health monitoring devices, biosensors, and wearable monitoring systems deployed to both first responders and patients. The system processes this data and visually assigns it to corresponding 2D or 3D avatars through integration with incident map generator 109a and incident database 109b. The implementation includes real-time visualization capabilities, generating animated displays of vital signs data through pulse oximetry and other monitoring techniques via UE 101 and associated wearable devices. This processed biometric data streams directly to UE 101 and DPTTS widget 103 for immediate access by emergency personnel.

The video capture system utilizes multiple recording devices including wearable cameras mounted on chest or head positions, aerial drone cameras, and other video recording hardware. The system supports multiple resolution capabilities from high definition through 8K recording, implementing various video formats including WebM, MPEG, AVI, and other standard protocols for maximum compatibility.

Position tracking functionality is achieved through multiple localization technologies including GPS, Bluetooth Low Energy, Wi-Fi, and cellular network triangulation systems. The generator processes this location data and transmits coordinate information to multiple system databases including navigation database 111b, biometrics database 110b, and incident database 109b to maintain comprehensive tracking capabilities.

The system implements comprehensive navigation functionality through incident navigator 111a. This navigation component maintains connectivity with navigation database 111b through communication network 107, while UE 101 establishes communication links to both the navigator and associated database.

The incident navigator 111a implements an advanced Points of Interest (POI) interface that renders interactive elements within the virtual environment. These POIs manifest as both two-dimensional and three-dimensional objects, appearing as digital overlays in various forms including circular indicators, pin-shaped markers, or balloon-style identifiers. The system enables dynamic interaction through clickable and animated POIs that respond to user engagement. Navigation occurs through a virtual camera system that provides immersive environmental visualization, complemented by teleportation capabilities that enable rapid movement between locations within the virtual incident space.

The POI system implements multiple specialized types to address various emergency response needs. Patient POIs serve as interactive hotspots that display real-time triage states according to the DIME system and integrate biometric data from the biometrics/position/video generator 110a. Video POIs enable direct playback of incident footage within the virtual environment generated by incident map generator 109a. The system further supports integration with third-party multimedia content from various streaming platforms and social media services. Additional POI types include accident markers for identifying emergency scenes and document POIs that provide access to various file formats including PDFs, HTML, and other standardized document types.

The navigation system implements a persistent interface structure through a sophisticated graphical user interface (GUI) that provides hierarchical access to both two-dimensional and three-dimensional content areas. This interface manifests either as an HTML layer surrounding the incident map, as an overlay atop the virtual environment, or as a mobile-optimized “hamburger” navigation menu. The system also supports direct integration of navigational elements within the three-dimensional environment through virtual signage implementations.

The system implements comprehensive patient tracking functionality through tagging manager 113a. This component maintains secure communication with tag database 113b via communication network 107, enabling real-time updates to the incident map and virtual environment displays on UE 101 and DPTTS widget 103.

The tagging manager implements a secure authentication framework requiring user credentials for system access. These authentication parameters are stored securely within tag database 113b, which utilizes advanced database architectures including SQL and NoSQL implementations. The system specifically supports MongoDB for document-oriented data storage, while maintaining compatibility with other NoSQL platforms including Azure Cosmos DB, Apache CouchDB, and additional enterprise database systems.

The scanning interface implementation provides multiple patient identification mechanisms through Near-Field Communication, Radio Frequency Identification, and optical barcode scanning capabilities. The system employs a sophisticated graphical targeting interface with real-time video feed integration to ensure precise scanning alignment. When processing physical triage tags, the tagging manager captures barcode data and records corresponding identifiers in tag database 113b.

Advanced computer vision algorithms enable automated extraction of patient information through ID card scanning capabilities. The system processes captured images using specialized CV algorithms to perform text extraction, capturing critical patient data including identification and contact information. Additionally, the implementation supports both passive and active RFID tag scanning, as well as NFC tag processing, enabling comprehensive patient tracking throughout emergency response operations. All captured tag identifiers and associated patient data are securely stored within tag database 113b for real-time access and analysis.

Building upon the tagging system capabilities, the system implements comprehensive data collection and analysis functionality through data collector and analyzer 115a. This component collects detailed information about first responders through multiple input mechanisms, including structured data forms presented via UE 101 and DPTTS widget 103. The system processes both identified and anonymous user data, with registration capabilities that capture essential personnel information while maintaining operational flexibility.

The implementation supports dynamic user assignment through Incident Commander Dashboard 114a, enabling real-time personnel tracking through permanent and semi-permanent identifiers. This architecture allows seamless integration of additional response personnel, such as supporting battalions or command structures, while maintaining continuous data collection throughout the registration process. The system tracks all interactions between first responders, patients, and incident elements, utilizing tagging manager 113a to associate relevant identifiers with each interaction point. Integration with biometrics/position/video generator 110a enables real-time access to patient vital signs and triage status through biometrics database 110b.

Data collector and analyzer 115a implements sophisticated data processing capabilities, storing collected information within collection database 115b. The analytical engine processes multiple data dimensions including temporal incident patterns (daily, weekly, monthly, and yearly aggregations), incident categorization, geographical distribution, and triage state distributions according to the DIME classification system. The system maintains continuous connectivity with tagging manager 113a to track patient transport status and first responder interactions throughout the incident lifecycle.

The visualization subsystem generates dynamic representations of analyzed data through configurable displays including graphs, charts, and animated gauges. These visualizations are integrated directly into incident commander dashboard 114a, providing real-time operational insights through an interactive digital interface. This comprehensive data processing and visualization capability enables effective incident management and resource allocation throughout emergency response operations.

The system implements a comprehensive command and control interface through incident commander dashboard 114a. This dashboard aggregates multiple data streams, integrating information from data collector and analyzer 115a, incident map generator 109a, biometrics/position/video generator 110a, incident navigator 111a, and tagging manager 113a into a unified operational display.

The system implements advanced visualization capabilities through multiple extended reality technologies. The incident commander dashboard generates holographic incident maps viewable through various display technologies, including direct view systems (LED, microLED, LCD, OLED) and specialized Head Mounted Displays. The implementation supports multiple HMD platforms including Meta Quest series, HTC Vive XR, Microsoft HoloLens, Magic Leap, Varjo XR, and Apple Vision Pro.

The dashboard incorporates sophisticated eye-tracking functionality through specialized hardware and software components. When implemented in physical environments, the system utilizes camera-based eye-tracking sensors to monitor commander attention patterns. This tracking system correlates gaze duration with specific data elements and video feeds, enabling precise measurement of commander engagement with particular information streams. The system processes this interaction data to optimize information presentation, automatically prioritizing displays based on commander attention patterns.

The data analysis subsystem implements advanced visualization capabilities including heat map generation to track first responder interactions within both virtual and physical environments. The system processes temporal interaction data to generate gradient maps indicating patient biometric trends. This capability enables early detection of patient health deterioration through trend analysis. The implementation further utilizes sophisticated machine learning algorithms, including convolutional neural networks and recurrent neural networks, to analyze biometric data patterns and predict potential adverse health events requiring immediate intervention.

The system architecture supports deployment across various operational scenarios beyond emergency response, maintaining functionality in workplace environments, sporting events, and educational settings while preserving core monitoring and analysis capabilities.

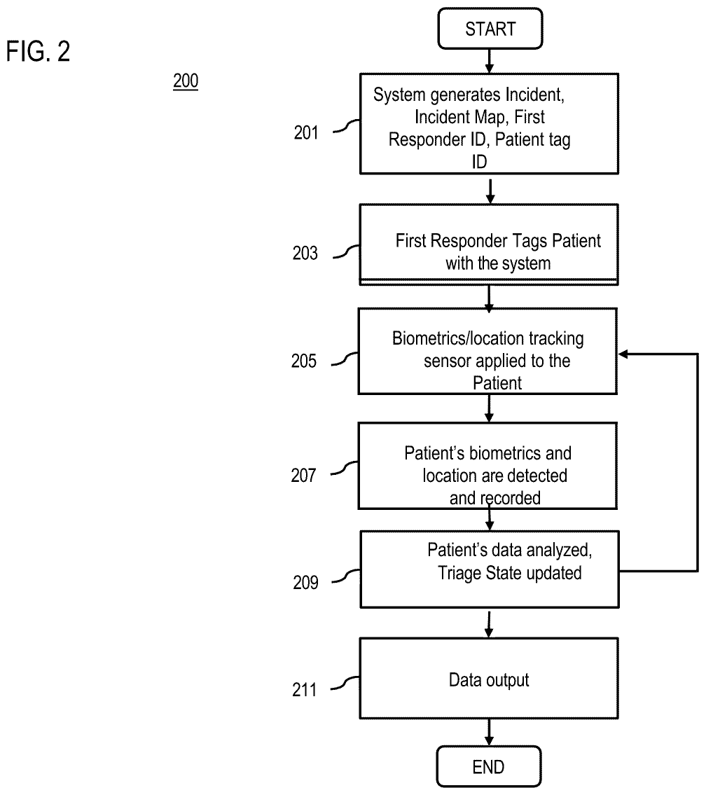

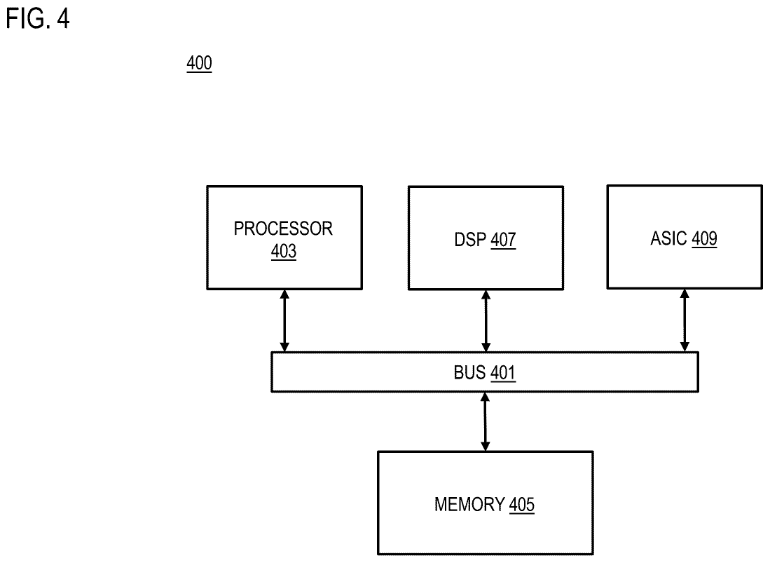

FIG. 2 illustrates a process 200 for digital patient triage and tracking according to an embodiment of the invention. The process 200 executes through a specialized chip set architecture comprising processor and memory components as detailed in FIG. 4.

At step 201, the system initiates incident response by generating core virtual environment components through incident map generator 109a. This includes creation of real-time 2D/3D virtual maps incorporating wireframe models, animations, textures, and virtual camera implementations. The generator accesses incident database 109b to retrieve environmental parameters and creates interactive Points of Interest for first responder engagement.

Step 203 implements patient registration and tagging functionality through a structured data collection interface. The system presents predetermined data fields for patient identification while supporting automated data capture through barcode and NFC scanning capabilities. The implementation includes anonymous patient tracking functionality, allowing the system to maintain continuity of care even when full patient identification is not immediately available.

At step 205, the system enables biometric monitoring through wearable sensor assignment. The implementation supports integration with commercial devices like Apple Watch for vital sign monitoring, while maintaining unique system-generated identifiers that correspond to patient records established in step 203. The system supports multiple activation methods including direct UI interaction through UE 101 and DPTTS widget 103, as well as patient-initiated opt-in through personal device integration.

Step 207 implements comprehensive biometric data collection, capturing vital signs including heart rate, blood pressure, ECG, and oxygen saturation levels. The system maintains unique identifiers for all patients and POIs, tracking first responder interactions through automated tagging and timestamp generation.

At step 209, the system processes collected patient data through analytical algorithms to determine appropriate triage states. The system implements a sophisticated DIME classification algorithm utilizing multiple data inputs including real-time biometric measurements, historical patient data analysis, environmental condition assessment, resource availability mapping, machine learning-based outcome prediction, and neural network-enabled triage optimization. The algorithm employs advanced statistical modeling and predictive analytics to determine optimal patient classification while maintaining adaptability to changing emergency conditions.

Step 211 implements comprehensive data output functionality with specific security and integration capabilities. The system outputs multiple data formats including raw collected data, processed analytical results, and combined data sets. The implementation supports standardized computer-readable formats enabling direct integration with electronic health records (EHR) systems, including comma-delineated data structures and human-readable formats like PDF and word documents. The system generates automated visualizations through dynamic dashboard interfaces displaying graphs, charts, and analytical plots. Critical to the implementation is HIPAA-compliant data de-personalization occurring pre-transmission, along with additional security protocols ensuring patient privacy protection.

The system architecture enables implementation through multiple technical approaches including software, specialized hardware components, and firmware configurations. The hardware implementation utilizes various processing components including: general processors for core system functions, Digital Signal Processing (DSP) chips for real-time data processing, application Specific Integrated Circuits (ASICs) for specialized operations, and Field Programmable Gate Arrays (FPGAs) for configurable processing capabilities This flexible architecture allows optimal deployment of system functionality across available computing resources while maintaining system performance and reliability.

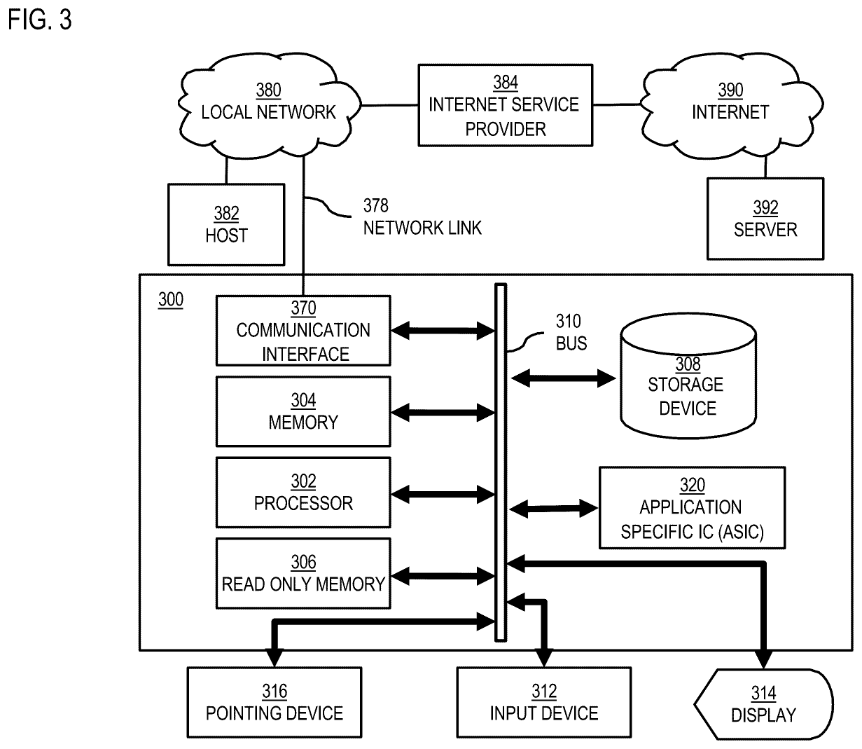

FIG. 3 illustrates a computer system 300 implementing specialized hardware and software components to enable digital triage and emergency response capabilities according to an embodiment of the invention. The system utilizes a communication bus 310 that facilitates information transfer between internal and external components through multiple data representation methods, including electrical voltages, magnetic fields, and quantum states. This architecture supports both binary (0,1) and quantum bit (qubit) data processing for advanced computational capabilities.

The processing architecture centers on processor 302, which executes specialized instruction sets through compiled computer programming code or direct machine language implementation. The processor performs critical operations including data comparison, position shifting, and logical operations (OR, XOR, AND) essential for emergency response calculations. The implementation supports various processor technologies including mechanical, electrical, magnetic, optical, chemical and quantum components.

The memory subsystem implements a multi-tiered architecture comprising memory 304 (RAM) for dynamic storage, ROM 306 for static instruction storage, and non-volatile storage device 308 for persistent data retention. Memory 304 enables independent access to stored information through memory addressing, while maintaining temporary execution values during processor operations.

The system incorporates specialized input/output devices including external input device 312 for user interaction and sensor data collection, display device 314 supporting multiple technologies (LED, OLED, CRT, LCD, plasma) for visualization, and pointing device 316 for interface control. Additional specialized hardware includes ASIC 320 for accelerated processing of graphics rendering, cryptographic operations, and medical equipment interfaces.

Communications interface 370 enables comprehensive network connectivity through multiple protocols and physical interfaces. The implementation supports parallel, serial, and USB connections, along with ISDN, DSL, and cable modem capabilities. The interface enables both wired (coaxial, fiber optic) and wireless (radio, infrared, optical) data transmission through network link 378 and local network 380.

The system utilizes various computer-readable media implementations including non-volatile storage (optical/magnetic disks), volatile memory, and transmission media (coaxial/fiber cables, electromagnetic waves). This comprehensive storage architecture supports robust data handling for emergency response operations while maintaining system reliability and data persistence.

FIG. 4 illustrates a chip set 400 upon which an embodiment of the invention may be implemented. Chip set 400 implements specialized processing capabilities through an integrated hardware architecture. The chip set incorporates processor and memory components described in FIG. 3 within consolidated physical packages that provide structural integrity, space optimization, and controlled electrical characteristics. The implementation supports single-chip integration for specific deployment scenarios.

The chip set architecture utilizes bus 401 as a primary communication mechanism between components. Processor 403 interfaces directly with bus 401 to execute instructions and process data stored in memory 405. The processing implementation supports multiple configurations including multi-core processing with two, four, eight or more cores operating independently; tandem microprocessor arrangements enabling instruction pipelining and multithreading; and/or specialized component integration including digital signal processors (DSP) 407 and application-specific integrated circuits (ASIC) 409

The DSP 407 implementation enables real-time signal processing independent of the main processor 403, while ASIC 409 handles specialized functions optimized for hardware execution. The architecture supports additional specialized components including field programmable gate arrays (FPGAs), controllers, and purpose-specific computing elements.

Memory 405 maintains connectivity to processor 403 and associated components through bus 401. The memory architecture implements both dynamic storage (RAM, magnetic/optical media) and static storage (ROM, CD-ROM) capabilities. This dual-mode memory system stores executable instructions for virtual environment generation while maintaining data generated during system operation.

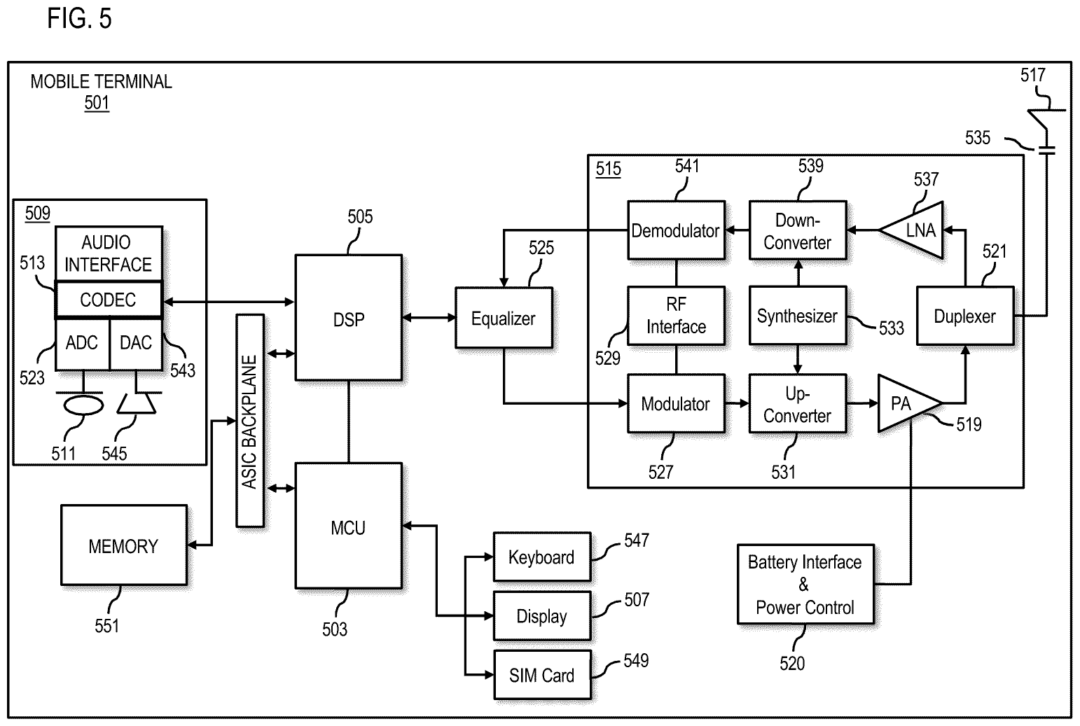

FIG. 5 illustrates a mobile terminal 501 representing a specific implementation of the User Equipment (UE) 101 within the digital triage and emergency response system 100. The mobile terminal 501 implements specialized hardware components that enable critical functionality within the digital triage and emergency response system. The terminal architecture comprises integrated front-end Radio Frequency circuitry and back-end baseband processing components, centered on Main Control Unit (MCU) 503, Digital Signal Processor (DSP) 505, and receiver/transmitter subsystems.

The mobile terminal implements a comprehensive signal processing chain beginning with audio capture through microphone 511 and associated amplification circuitry, routing through CODEC 513 for digital signal processing. Radio section 515 manages power amplification and frequency conversion via antenna 517, with power amplifier 519 providing signal enhancement under MCU 503 control. Power management occurs through the battery interface and control unit 520.

Signal processing implementation occurs through multiple stages, beginning with analog-to-digital conversion via ADC 523, followed by DSP 505 processing for encoding, encryption, and protocol formatting. The system supports multiple wireless protocols including EDGE, GPRS, GSM, IMS, UMTS, WiMAX, LTE, CDMA, and WiFi, enabling comprehensive emergency response communications. Signal processing continues through equalizer 525 for transmission compensation, followed by modulator 527 and RF interface 529 for carrier wave generation.

The transmission architecture incorporates up-converter 531 and synthesizer 533 for frequency preparation, with final stage amplification through PA 519. The receiving subsystem implements LNA 537 for initial amplification, down-converter 539 for carrier removal, and demodulator 541 for digital stream extraction. Processed signals route through equalizer 525 and DSP 505 before final conversion via DAC 543 for audio output through speaker 545.

System control centers on MCU 503, which manages keyboard 547 input and display 507 output while coordinating with DSP 505 for signal processing operations. The implementation includes SIM card 549 support for network authentication and memory 551 for data storage. The memory architecture supports multiple storage technologies including RAM, flash memory, and non-volatile storage options for maintaining system and operational data.

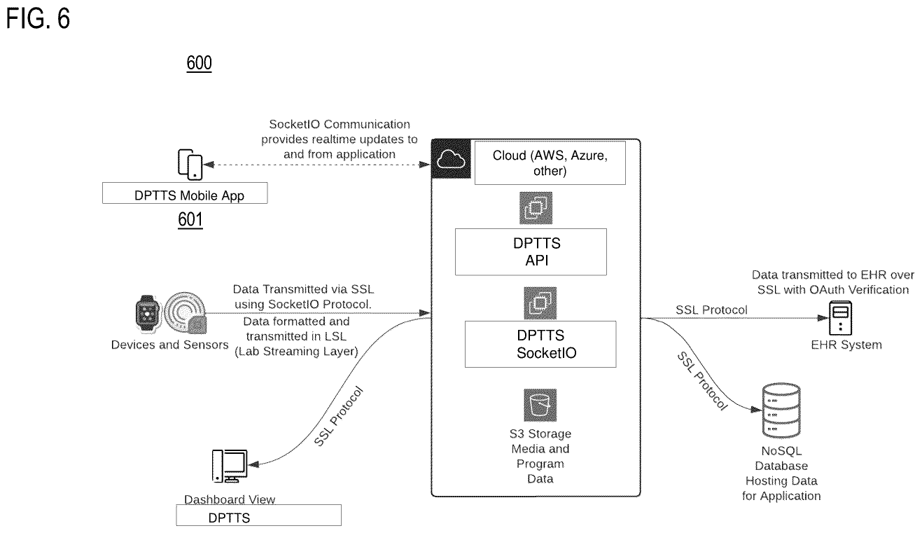

FIG. 6 illustrates a system architecture 600 for implementing the Digital Patient Triage and Tracking System (DPTTS) according to an embodiment of the invention. The architecture comprises multiple integrated components that enable secure real-time data transmission and processing for emergency response operations. The system implements a mobile application layer through DPTTS Mobile App 601, which connects to cloud infrastructure (AWS, Azure, or similar platforms) through multiple secure communication channels. The implementation utilizes SocketIO Communication protocols to enable real-time bidirectional updates between the application and server components.

The security architecture implements end-to-end encryption, multi-factor authentication, blockchain-based audit trails, and quantum-resistant cryptography. The system exceeds HIPAA compliance requirements while enabling secure data exchange across emergency response networks.

The implementation includes specialized data formatting through Lab Streaming Layer (LSL) protocols. This enables standardized handling of biometric and sensor data collected from various devices and monitoring equipment. The architecture maintains dedicated storage media and program data repositories, while utilizing NoSQL database implementations for application data hosting.

The system provides multiple user interface points, including a comprehensive dashboard view through the DPTTS interface. This dashboard enables real-time visualization of emergency response data while maintaining secure connections to backend services through SSL protocols. The architecture supports integration with various devices and sensors through standardized communication protocols, enabling comprehensive data collection and monitoring capabilities.

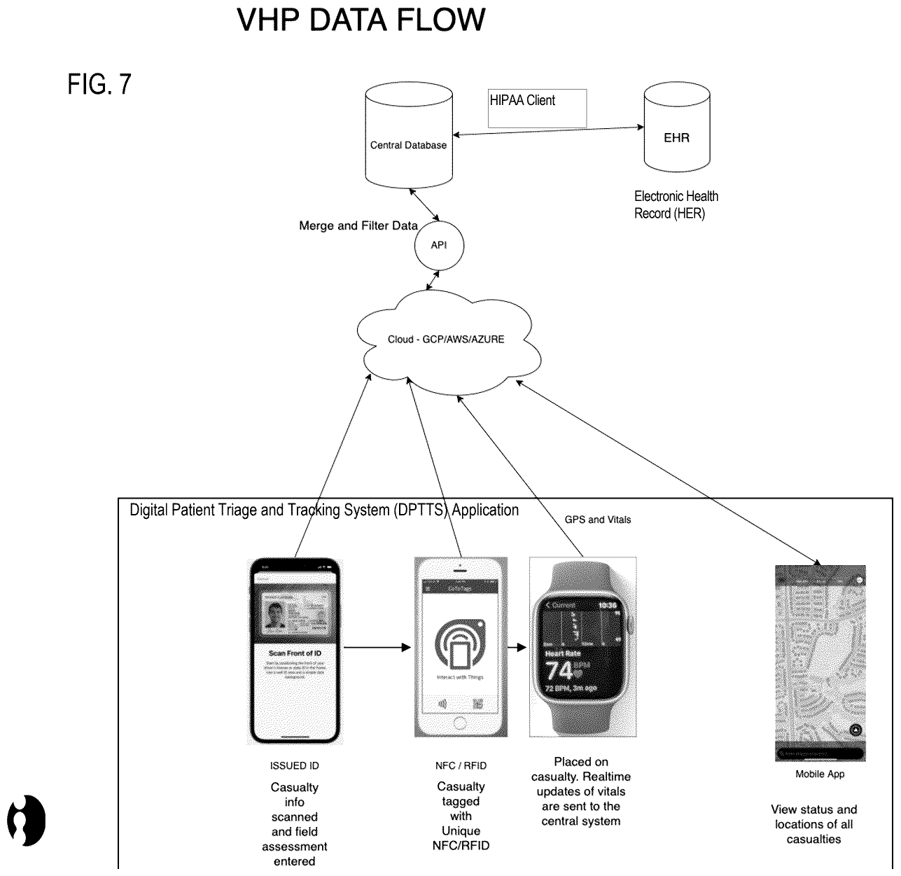

FIG. 7 illustrates a data flow architecture for implementing secure patient information handling within the DPTTS. The implementation establishes a multi-layered approach to data processing and transmission while maintaining HIPAA compliance. The system architecture begins with field-level data collection through multiple input channels. First responders utilize NFC/RFID technology to uniquely tag casualties, enabling continuous patient tracking throughout the emergency response process. The implementation captures casualty information through scanning operations and field assessments, which are then securely transmitted through the system.

The architecture implements real-time vital sign monitoring through specialized biosensors applied to casualties. These sensors continuously transmit updates to the central system, enabling dynamic patient status monitoring. The mobile application interface provides comprehensive visualization capabilities, allowing responders to view status and locations of all casualties in real-time.

Data processing occurs through multiple secure stages. The system implements a central database that processes and filters incoming data streams before routing information to the Electronic Health Record (EHR) system. This filtering mechanism ensures appropriate data handling while maintaining HIPAA compliance requirements.

The implementation utilizes cloud infrastructure (GCP/AWS/AZURE) to host the DPTTS application, enabling scalable processing capabilities while maintaining secure data transmission pathways. The architecture incorporates specialized APIs for managing data flow between system components, ensuring reliable communication between field devices and central processing systems.

The system maintains secure connectivity with healthcare information systems through HIPAA-compliant interfaces. A central database manages data merging and filtering operations, ensuring appropriate information handling while enabling efficient access to critical patient data through the Digital Patient Triage and Tracking System application.

The system implements specialized navigation and interface components through multiple integrated subsystems. The Points of Interest (POI) interface provides distinct implementations for tracking emergency response personnel, patients, and incident locations. The First Responder POI implementation enables real-time position tracking and biometric data display, incorporating geolocation coordinates, unit identification, vital sign monitoring, and status indicators.

The Patient POI implementation provides comprehensive tracking capabilities through real-time location monitoring, patient identification systems, biometric data visualization, and triage state indication. The Disaster POI implementation manages incident mapping through environmental hazard tracking, incident type classification, geographic coordinate mapping, and situational parameter monitoring.

The administrative portal implements a web-based interface utilizing HIPAA-compliant security protocols. The frontend architecture incorporates secure login authentication, emergency event management, user access control, hotspot configuration tools, and content management systems. The backend utilizes NoSQL database implementation supporting document-oriented storage, key-value pair management, column-oriented data structures, and access control mechanisms.

The analytics subsystem implements advanced monitoring capabilities through integrated camera systems for eye tracking and facial expression analysis. The system processes three-dimensional heat-mapping for user interaction analysis while maintaining integration with standard analytics platforms and real-time user interaction monitoring.

The communication system implements bidirectional data exchange through video conferencing capabilities, text-based messaging, voice communication channels, real-time position tracking, and multimedia content sharing. The wearable sensor system implements inertial measurement capabilities through IMU-based position tracking, visual-inertial odometry processing, indoor navigation algorithms, and GPS-denied environment operations. The system implements precise indoor navigation through multiple complementary technologies: visual-inertial odometry processing, ultra-wideband ranging, magnetic field mapping, WiFi fingerprinting, sensor fusion algorithms, and dead reckoning capabilities. The implementation maintains sub-meter accuracy while operating independently of external positioning systems.

All implementations maintain HIPAA compliance through specialized security protocols and data handling procedures while enabling seamless integration with existing emergency response infrastructure. The system architecture supports continuous monitoring and data exchange between field operations and command centers while maintaining patient privacy protections.

The system maintains operational continuity through active-active failover, geographic distribution, data replication, and comprehensive disaster recovery protocols. The implementation ensures uninterrupted emergency response capabilities while protecting critical patient information.

The database architecture implements a comprehensive data model incorporating patient information structures, biometric data schemas, geospatial indexing, and temporal databases. The system utilizes RESTful and GraphQL APIs supporting real-time data streaming, batch processing, and event-driven architecture while maintaining system modularity.

The implementation maintains strict operational benchmarks including sub-second response times, 99.999% uptime, real-time data synchronization, and network latency optimization. The system architecture enables continuous monitoring and performance optimization while ensuring reliable emergency response operations.

While the invention has been described in connection with a number of embodiments and implementations, the invention is not so limited but covers various apparent modifications and equivalent arrangements, which fall within the purview of the appended claims. Although features of the invention are expressed in certain combinations among the claims, it is contemplated that these features can be arranged in any combination and order. The invention has been described herein using specific embodiments for illustrative purposes only. It will be readily apparent to one of ordinary skill in the art, however, that the principles of the invention can be embodied in other ways. Therefore, the invention should not be regarded as limited in scope to the specific embodiments disclosed herein; it should be fully commensurate in scope with the following claims.

We claim:

1. A system for digital emergency triage and tracking, comprising:
an incident map generator configured to generate a real-time digital map of an emergency incident;
a biometrics generator configured to collect and transmit one or more vital signs of one or more patients;
an incident navigator configured to provide a points of interest (POI) interface;
a tagging manager configured to associate digital identifiers with the one or more patients; and
a data collector and analyzer configured to process and analyze the vital signs of the one or more patients, wherein the system is configured to digitally track and monitor triage states and locations of the one or more patients.

2. The system of claim 1, wherein the incident map generator is configured to generate a model of an emergency environment using geographical information system (GIS) data.

3. The system of claim 1, wherein the one or more vital signs are selected from the group consisting of: heart rate, blood pressure, oxygen saturation, respiration rate, electrocardiogram, electroencephalogram, body temperature, and a combination thereof.

4. The system of claim 1, wherein the POI interface is configured to display a virtual representation of an emergency location, the one or more patients, and one or more first responders.

5. The system of claim 1, wherein the digital identifiers are associated with physical triage tags.

6. The system of claim 1, wherein the data collector and analyzer is configured to:
determine the triage states of the one or more patients.

7. The system of claim 1, further comprising an incident commander dashboard configured to display aggregated data associated with the one or more patients and the real-time digital map of the emergency incident data using at least one of augmented reality, virtual reality, or mixed reality technologies.

8. A method for digital emergency triage and tracking, comprising:
generating a virtual incident environment;
registering and tagging a plurality of patients with digital identifiers;
collecting vital signs using wearable sensors of the plurality of patients;
analyzing the collected vital signs to determine triage states of the plurality of patients;
tracking locations of the plurality of patients; and
outputting the collected vital signs and the determined triage states to an emergency care facility.

9. The method of claim 8, wherein generating the virtual incident environment comprises:
accessing geographical information system data;
generating a two-dimensional or three-dimensional model;
adding one or more virtual points of interest; and
providing a navigation interface.

10. The method of claim 8, wherein collecting vital signs using wearable sensors of the plurality of patients comprises measuring at least one of: heart rate, blood pressure, oxygen saturation, respiration rate, electrocardiogram, electroencephalogram, body temperature.

11. The method of claim 8, wherein registering and tagging the plurality of patients comprises:
scanning physical triage tags;
capturing patient identification information; and
storing patient data in a database.

12. The method of claim 8, wherein analyzing the collected vital signs to determine triage states comprises:
processing the collected vital signs;
determining triage states using predefined algorithms;
generating data visualizations; and
updating patient status in real-time.

13. The method of claim 8, further comprising displaying aggregated incident data through an incident commander dashboard using extended reality technologies.

14. A non-transitory computer-readable medium storing instructions that, when executed by a processor, cause the processor to perform operations comprising:
generating a virtual incident environment;
managing digital patient triage tags;
collecting and analyzing patient vital signs;
tracking patient locations; and
providing real-time incident awareness.

15. The computer-readable medium of claim 14, wherein the operations further comprise:
displaying patient vital signs on virtual avatars;
updating triage states in real-time;
generating heat maps of patient data; and
providing predictive analytics using machine learning.

16. The computer-readable medium of claim 14, wherein managing digital patient triage tags comprises:
scanning physical triage tags;
associating digital identifiers with patients;
storing triage states; and
updating patient status.

17. A wearable device for patient monitoring during emergency response, comprising:
one or more biometric sensors configured to measure one or more patient vital signs;
a location tracking module;
a wireless communication interface; and
a processor configured to transmit patient data to a digital triage system.

18. The wearable device of claim 17, wherein the one or more biometric sensors comprise at least one of: a heart rate sensor; a blood pressure sensor; an oxygen saturation sensor; a respiration rate sensor; and a temperature sensor.

19. A system for emergency incident command, comprising:
a holographic display interface;
a virtual incident map;
real-time patient tracking;
biometric data visualization; and
predictive analytics capabilities.

20. The system of claim 19, further comprising:
eye-tracking hardware configured to monitor commander interactions;
heat mapping functionality for patient data analysis; and
machine learning algorithms for health event prediction.
